(19) United States
(12) Patent Application Publication
(10) Pub. No.: US 2021/0267472 A1
(43) Pub. Date: Sep. 2, 2021

(54) HEART RATE VARIABILITY ANALYSIS IN MAMMALIANS

(71) Applicant: TECHNION RESEARCH & DEVELOPMENT FOUNDATION LIMITED, Haifa (IL)

(72) Inventors: Yael YANIV, Haifa (IL); Joachim BEHAR, Haifa (IL); Aviv ROSENBERG, Kiryat Tivon (IL)

(21) Appl. No.: 17/259,172

(22) PCT Filed: Jul. 11, 2019
(86) PCT No.: PCT/IL2019/050785

§ 371 Date: Jan. 10, 2021

Related U.S. Application Data
(60) Provisional application No. 62/697,043, filed on Jul. 12, 2018.

(51) Int. Cl. A61B 5/024; A61B 5/352; A61B 5/366; A61B 5/00; A61B 5/388; A61B 5/36

(52) U.S. Cl. CPC A61B 5/02405; A61B 5/02416; A61B 5/352; A61B 2503/40; A61B 5/7214; A61B 5/388; A61B 5/36; A61B 5/366

(57) ABSTRACT

A system comprising at least one hardware processor; and a non-transitory computer-readable storage medium having stored thereon program instructions, the program instructions executable by the at least one hardware processor to: receive, as input, a signal representing temporal beat activity in a mammalian, receive indication of user selection of an integration level of said beat rate activity, receive indication of user selection of a species of said mammalian, and determine heart rate variation (HRV) in said signal, based, at least in part, on said type of said beat rate activity and said species of said mammalian.

### CROSS REFERENCE TO RELATED APPLICATIONS

This application claims the benefit of priority of U.S. Provisional Patent Application No. 62/697,043 filed on Jul. 12, 2018, entitled “EART RATE VARIABILITY ANALYSIS IN MAMMALIANS”, the contents of which are incorporated herein by reference in their entirety.

### FIELD OF THE INVENTION

The invention relates to methods for monitoring of beat rate variability.

### BACKGROUND

The time variation between consecutive heartbeats is commonly referred to as heart rate variability (HRV). Studies have shown that measures quantifying HRV together with heart rate (HR) can provide useful information on cardiovascular health. Use cases include prediction of sepsis in neonates, detection of atrial fibrillation, detection of obstructive sleep apnea, or identifying intrauterine growth restricted fetuses. Importantly, loss of complexity in the HRV of a patient with cardiovascular disease has been correlated with an increase in both morbidity and mortality. However, the mechanisms that control HRV are not well understood nor is it known whether and how HRV patterns might be an indicator of functional or structural heart diseases. Studying HRV in animals may provide the key to investigating this question.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive, as input, a signal representing temporal beat activity in a mammalian, receive indication of user selection of an integration level of said beat rate activity, receive indication of user selection of a species of said mammalian, and determine heart rate variation (HRV) in said signal, based, at least in part, on said integration level and said species of said mammalian.

There is also provided, in an embodiment, a method comprising operating at least one hardware processor for executing a genetic-type machine learning algorithm configured for: a non-transitory computer-readable storage medium having stored thereon program instructions, the program instructions executable by the at least one hardware processor to: receiving, as input, a signal representing temporal beat activity in a mammalian, receiving indication of user selection of an integration level of said beat rate activity, receiving indication of user selection of a species of said mammalian, and determining HRV in said signal, based, at least in part, on said integration level and said species of said mammalian.

There is further provided, in an embodiment, a computer program product comprising a non-transitory computer-readable storage medium having program instructions embodied therewith, the program instructions executable by at least one hardware processor to execute a genetic-type machine learning algorithm configured for: receiving, as input, a signal representing temporal beat activity in a mammalian, receiving indication of user selection of an integration level of said beat rate activity, receiving indication of user selection of a species of said mammalian, and determining HRV in said signal, based, at least in part, on said integration level and said species of said mammalian.

In some embodiments, said integration level is selected from the group consisting of: in vivo heart beat rate, sinoatrial node tissue beat rate, and sinoatrial node cell beat rate.

In some embodiments, said signal is at least one of an action potential signal, an electrogram signal, an electrocardiograph (ECG) signal, and a photoplethysmogram (PPG) signal.

In some embodiments, said determining is based, at least in part, on one or more HRV analysis methods selected from the group consisting of: power spectral density analysis, time domain analysis, frequency domain analysis, nonlinear analysis, detrended fluctuation analysis, sample entropy analysis, and fragmentation HRV analysis.

In some embodiments, said determining is further based, at least in part, on one or more HRV parameters associated with said species of said mammalian. In some embodiments, one or more values associated with each of said parameters is adapted based, at least in part, on said species of said mammalian.

In some embodiments, said HRV parameters are selected from the group consisting of: pNNxx threshold, very low frequency (VLF) band, low frequency (LF) band, high frequency (HF) band, and window size for power spectral density (PSD).

In some embodiments, at least some of said HRV parameters associated with said species of said mammalian are retrieved from a database of said HRV parameters associated with a plurality of mammalian species.

In some embodiments, at least some of said HRV parameters with respect to a mammalian species are calculated based, at least in part, on an empirical relationship between a mean heart rate value for said species and a power spectral density analysis of said beat rate activity.

In some embodiments, said determining comprises first detecting R-peaks in said signal, based, at least in part, on detecting the R-peak location within a specified portion of a duration of each QRS complex in said signal, wherein said detecting takes into account one or more physical parameters associated with a species of said mammalian. In some embodiments, said specified portion is between 75% and 85% of an average said duration of said QRS complex associated with said species of said mammalian.

In some embodiments, said physical parameters are selected from the group consisting of: mean heart rate, mean QRS duration, mean QT interval duration, minimum R-peak-to-R-peak interval, maximum R-peak-to-R-peak interval, typical QRS peak-to-peak amplitude, and minimum QRS peak-to-peak amplitude.

In some embodiments, said detecting further comprises filtering said signal to remove all said beats associated with non-normal sinus node polarizations. In some embodiments, said filtering is based on one or more of: range-based filtering, moving average filtering, and quotient filtering.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 illustrates beat rate integration levels;

FIG. 2 is a block diagram of an exemplary system for analyzing heart rate variation (HRV) in mammalians, according to an embodiment;

FIGS. 3A-3B illustrate representative examples of R-peaks detection results, according to an embodiment;

FIGS. 4A-4B illustrate examples of noise leading to inaccurate R-peak detection, according to an embodiment;

FIG. 4C illustrates a prefiltering step, according to an embodiment;

FIG. 5 is a block diagram summarizing the main steps to identify frequency bands, according to an embodiment;

FIGS. 6A-6D show Gaussian mixture models estimated for various mammals, according to an embodiment;

FIGS. 7A-7C show the relative power of each band over the total power for each mammal, according to an embodiment;

FIGS. 8A-8C show the scaling relation exists between the HR and the dominant PSD peak in each of the frequency bands, according to an embodiment;

FIG. 9 shows the result of the line fit for the scaling relation exists between the HR and the dominant PSD peak in each of the frequency bands, according to an embodiment;

FIGS. 10A-10C show allometric scaling between BMₘ and the mean of the Gaussians describing each band, according to an embodiment; and

FIG. 11 illustrates aliasing measurements associated with the Lomb method, according to an embodiment.

### DETAILED DESCRIPTION

Disclosed herein are a system, method, and computer program product for analyzing heart rate variability (HRV) in mammalians. The present invention may be user-configurable for measuring HRV in humans as well as multiple other mammalian species, at several integration levels, including at a sinoatrial cell level, a sinoatrial tissue level, and/or a whole heart level.

As noted above, HRV analysis can reveal information about the underlying physiological processes that control its dynamics. For example, a loss of complexity in HRV has been documented in several cardiovascular diseases and has been correlated with an increase in morbidity and mortality, while abnormal beating patterns can be characteristic of arrhythmias, such as atrial fibrillation. Currently, several species of mammals are used for cardiovascular research. Dogs, rabbits and mice have been of particular interest: dogs are physiologically close to humans and thus a reliable experimental model to study cardiac diseases. The rabbit is the smallest mammal with Ca²⁺ dynamics similar to humans. With the recent advances in genome manipulation technologies, there has been increased interest in using animals with mutations designed to overexpress or knock out genes implicated in human cardiovascular diseases. Mice have been of particular interest in that regard. However, to date, there are no standard tools for HRV analysis of mammalian electrocardiogram (ECG) data.

Accordingly, in some embodiments, the present invention may be configured for providing traditional HRV analysis with respect to multiple non-human mammalian species, based upon a plurality of physiological parameters associated with each mammalian species. In some embodiments, the present invention adapts known HRV analysis methods to non-human mammals, based upon empirical results of measurements in various mammalian species.

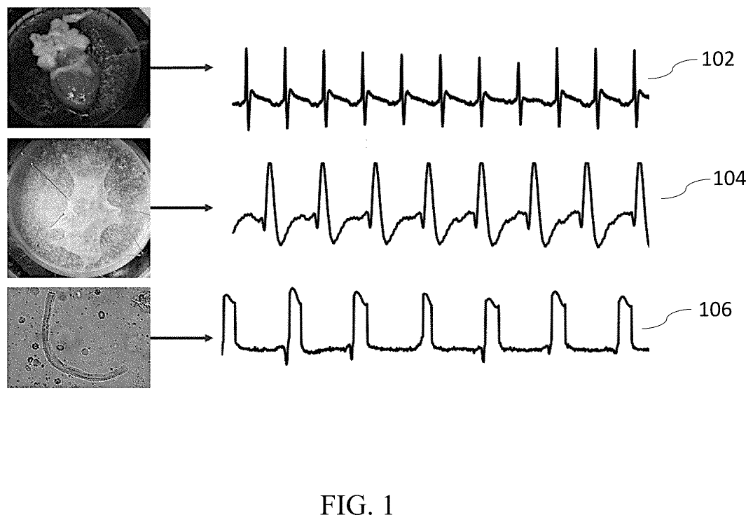

In some embodiments, the present invention may be configured for performing such HRV analysis at multiple levels of integration. For example, as shown in in FIG. 1, the present invention may be configured for receiving as an input an electrophysiological base signal 102 representing whole heart beat rate, sinoatrial tissue beat rate 104, and/or sinoatrial cell beat rate 106. In some embodiments, the electric signal may be an action potential signal, an electrogram signal, and/or an electrocardiograph (ECG) signal. In other embodiments, the signal may be any pulsatile signal representing temporal beat activity, such as photoplethysmogram (PPG) or any other similar signal.

In some embodiments, the present invention creates a user-friendly software platform for HRV analysis in different mammalians. The software platform computes traditional HRV measures with parameters adapted to each mammalian species, and outputs standard HRV measures and visualization plots for analysis. Because the software platform is open source, it is easy to add additional HRV measures to the software, or adapt it to any research-specific usage. In addition, the software platform may have a number of novel functionalities not available in existing HRV software. For example, it may allow manual annotation of the signal quality and correction of mis-detected R-peaks. It may also allow the HRV analysis to be performed on multiple segments (batch processing), thus making it possible to track changes in HRV measures over time for a given record.

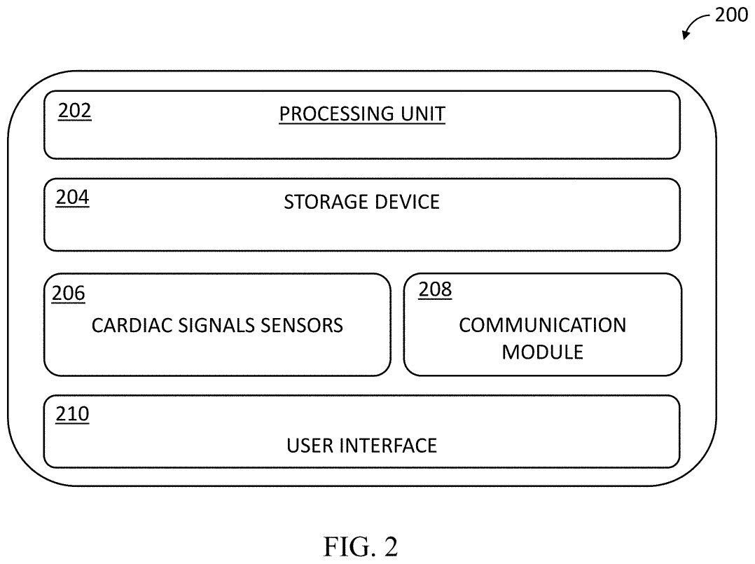

With reference to FIG. 2, in some embodiments, an exemplary system 200 in accordance with the present disclosure comprises processing unit 202, comprising at least one hardware processor. Processing unit 202 is communicatively connected to a non-transient computer readable storage device 204. Processing unit 202 is configured to automatically control the operation of system 200, based on one or more applications, sets of software instructions, and algorithms stored storage device 204. Storage device 204 may be configured to have stored thereon digital representations of electrophysiological signals and/or measurements. In some embodiments, storage device 204 may have stored thereon a database comprising multiple sets of standardized electrophysiological data from a plurality of mammalian species. In some embodiments, processing unit 202 is configured to receive and process one or more electrophysiological signals and/or measurements collected by one or more sensors 206. System 200 may further comprise a communication module 208, which may be configured to connect system 200 through a communication network, such as the Internet, a local area network, a wide area network, and/or a wireless network. For example, system 200 may be configured to receive electrophysiological signals from a remote acquisition unit, and/or export measurement results over a network via communication module 208. In some embodiments, system 200 comprises a user interface 210 which allows, e.g., a user to configure system 200 to one of multiple input signal types and mammalian species. User interface 210 may also be configured to allow the user to perform manual corrections and/or annotations on the measurement results. System 200 described herein is only an exemplary embodiment, and may have more or fewer components than shown, may combine two or more components, or a may have a different configuration or arrangement of the components. The various components of system 200 may be implemented in hardware, software, or a combination of both hardware and software. According to various other embodiments, processing unit 202 or processing tasks performed thereby may be implemented by a handheld or worn computing device such as, but not limited to, a smart phone, a tablet computer, a notepad computer, and the like. In addition, aspects of the present system which can be implemented by computer program instructions, may be executed on a general-purpose computer, a special-purpose computer, or other programmable data processing apparatus.

In some embodiments, the present invention, which may be implemented as exemplary system 200, may be configured to provide an automated R-peak detector. In some embodiments, the R-peak detector may be user-configurable to detect R-peaks in beat rate data related to humans as well as non-human mammalian species. In some embodiments, the R-peak detector is based, at least in part, on one or more known QRS complex detection methodologies (see, e.g., Johnson, A. E. W., Behar, J., Andreotti, F., Clifford, G. D., and Oster, J. (2015). Multimodal heart beat detection using signal quality indices. Physiological Measurement 36, 1665. doi:10.1088/0967-3334/36/8/1665; Llamedo, M., and Martinez, J. (2014). QRS detectors performance comparison in public databases. Computing in Cardiology Conference 41, 357-60; Pan, J., and Tompkins, W. J. (1985). A Real-Time QRS Detection Algorithm. IEEE Transactions on Biomedical Engineering 32, 230-6. doi:10.1109/TBME.1985.325532).

One such known QRS complex detection tool is the gqrs detector, part of the PhysioNet WFDB toolkit which has been shown to perform very accurately on several ECG databases (see Goldberger, A. L., Amaral, L. A., Glass, L., Hausdorff, J. M., Ivanov, P. C., Mark, R. G., et al. (2000). PhysioBank, PhysioToolkit, and PhysioNet: components of a new research resource for complex physiologic signals. Circulation 101, E215-20. doi:10.1161/01.CIR.101.23.e215). The gqrs tool can further be configured to work on non-human data by changing its configuration parameters. In its current configuration, the gqrs tool detects the beginning of the QRS complex, but not the R-peak itself. However, for HRV calculation, beat-to-beat analysis of the R-peak provides a more stable fiducial point than the QRS onset.

Accordingly, in some embodiments, the present invention provides for an R-peak detector, termed rqrs, which uses the QRS complex detection capabilities of gqrs for detecting R-peaks, based on searching forward for R-peaks in a small window from the start time of each QRS complex detection. In some embodiments, the maximal value detected in this time window is indicative of the R-peak of the QRS complex. In some embodiments, the duration of the window is configured to be between 75%-85% of the average duration of one QRS complex of the relevant species.

To make sure rqrs performs robust R-peak detections, rqrs was tested on the updated 2014 PhysioNet Challenge training set (see Silva, I., Moody, B., Behar, J., Johnson, A., Oster, J., Clifford, G. D., et al. (2015). Robust detection of heart beats in multimodal data. Physiological Measurement 36, 1629-44. doi:10.1088/0967-3334/36/8/1629). This set contains 200 ECG recordings, some of which are very challenging for R-peak detection algorithms due to noise and artifacts. All ECG recordings in the set have reference, manually corrected, R-peak annotations. The results of a comparison between the results of gqrs and rqrs on the PhysioNet 2014 Challenge database can be seen in table 1 below. To assess the R-peak detection accuracy, the following standard statistical measures were used:
Sensitivity (Se) is the fraction of correctly detected events (R-peaks),

$$S e = \frac{T P}{T P + F N} ,$$

where TP (true positive) is the number of detections that have a matching reference annotation, FP (false positive) is the number of detections that do not have a matching reference annotation (incorrect detections), and FN (false negative) is the number of reference annotations that were not matched with a detection (missed events).
Positive predictive value (PPV) is the fraction of detections that were actual events (R-peaks):

$$P P V = \frac{T P}{T P + F P} .$$

The overall detection accuracy measure, F₁ is then defined as:

$${F}_{1} = 2 \cdot \frac{PPV \cdot Se}{P P V + S e} .$$

The F₁ statistic was used for assessing the performance of R-peak detection algorithms due to its ability to combine multiple fractional measures by using a harmonic mean between the Se and PPV. To assess an R-peak detector's performance, the detected beat locations were compared to reference manual annotations for that record. For humans, the detection times were compared to the reference annotations using a 150 ms tolerance window around each detection as standardized by ANSFAAMI. This tolerance window needs to be mammal-dependent to account for the variable HR range across mammals. For each mammal, there was defined the tolerance window as 150 ms times the ratio between the human to mammal mean HR. This led to tolerance window values of 90 ms, 40 ms and 17 ms for dogs, rabbits, and mice, respectively. These tolerance windows were used to compute the Se, PPV and F₁ statistics for assessing the R-peak detector accuracy for the different mammals. As can be seen in table 1, rqrs performs similarly to gqrs, with a slightly better overall score, arguably due to the fact that it finds the R-peaks themselves, increasing the chance that a reference annotation will be within the comparison window around the detection.

TABLE 1
Performance comparison of gqrs and rqrs

| | Mean | | Gross | | |
|---|---|---|---|---|---|
| | Se | PPV | Se | PPV | F₁ |
| gqrs | 94.409 | 92.615 | 94.665 | 90.976 | 92.784 |
| rqrs | 93.809 | 93.624 | 93.845 | 93.026 | 93.434 |

In some embodiments, the rqrs R-peak detector of the present invention is user-configurable for detecting R-peaks in non-human mammalian data, based on one or more parameters. These parameters may include one or more of:
HRₘ, the typical heart rate of the mammal, taken as being the mean HR;
QSₘ, the typical QRS duration, taken as being the mean QRS duration;
QTₘ, the typical QT interval duration, taken as being the mean QT duration;
RRₘᵢₙ, the minimum RR interval;
RRₘₐₓ, the maximum RR interval;
QRSₐ the typical QRS peak-to-peak amplitude; and
QRSₐₘᵢₙ, the minimum QRS peak-to-peak amplitude.

In some embodiments, these parameters may be obtained, at least in part, based upon previous measurements conducted with respect to the relevant species. For example, in table 2 below, the parameters for humans were taken from the default PhysioNet configuration file. Dog, rabbit and mouse data were taken from physiological measurements reported in other studies (Chapel et al., 2017; Cintra et al., 2017; Hanton and Rabemampianina, 2006; Lord et al., 2010; Sysa-Shah et al., 2015) and in the Research Animal Resources Center database (University Wisconsin-Madison, 2018).

TABLE 2
Exemplary Mammalian R-peak Parameters

| | | Values | | | |
|---|---|---|---|---|---|
| Parameter | Description | Human | Dog | Rabbit | Mouse |
| HRₘ | Typical heart rate (beats/min) | 75 | 109.5 | 264 | 608 |
| QSₘ | Typical QRS duration (sec) | 0.07 | 0.04 | 0.02 | 0.00718 |
| QT | Typical QT interval duration (sec) | 0.35 | 0.19 | 0.12 | 0.03 |
| RRₘᵢₙ | Minimum RR interval (“refractory | 0.28 | 0.25 | 0.14 | 0.05 |
| | period”) (sec) | | | | |
| RRₘₐₓ | Maximum RR interval (sec) | 2.4 | 1.2 | 0.58 | 0.24 |
| QRS_{α} | Typical QRS peak-to-peak amplitude | 750 | 1120 | 294 | 1090 |
| | [μV] | | | | |
| QRSₐₘᵢₙ | Minimum QRS peak-to-peak | 130 | 100 | 114 | 370 |
| | amplitude [μV] | | | | |

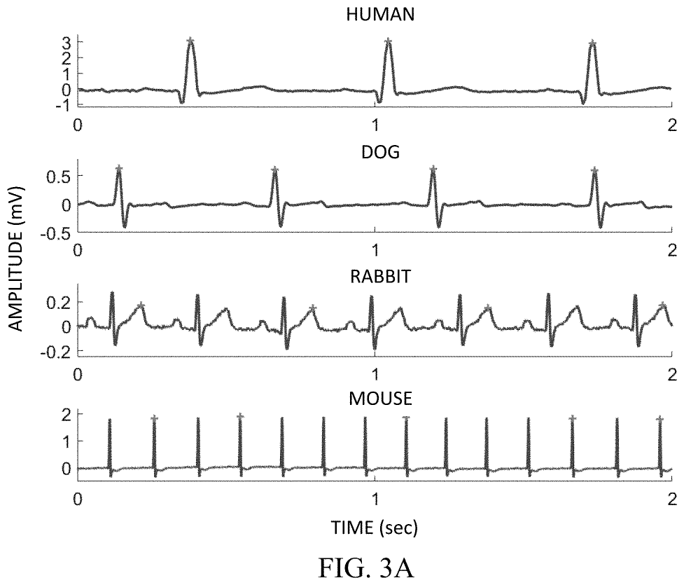

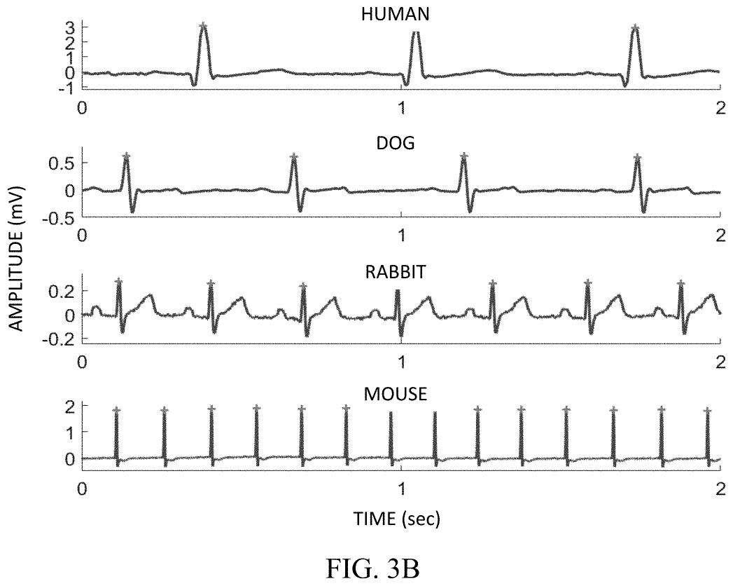

FIGS. 3A-3B show a representative example of the R-peaks detected using gqrs and rqrs, respectively, on the same 2-second segment of data. As can be seen, the performance of rqrs with respect to some species (e.g., rabbit, mouse) is significantly more consistent than that of gqrs.

In some embodiments, after performing R-peak detection, the present system is then configured for performing pre-processing with respect to the R-peak detection results. It should be noted that the basic interval lengths between R-peaks can be obtained simply by measuring the time differences between consecutive R-peaks (RR intervals). However, only beats resulting from normal sinus node depolarizations (i.e., not arrhythmic, paced, ventricular, etc.) should be used for HRV metric calculations. The intervals between such normal beats are referred to as NN intervals. Accordingly, in some embodiments, the present system is configured for filtering out so-called ectopic beats, a type of premature beat that is quite common in ECG signals and often originates from the atrioventricular node or pacemakers in the ventricles, due to a blockage of the depolarization signal from the sinoatrial node (SAN). Rejecting ectopic beats in turn will allow the isolation of the contribution of the SAN to the HRV. In addition to ectopic beats, in some embodiments, the present system may be configured for rejecting various artifacts caused, e.g., by movement of the subject or by the recording equipment.

In some embodiments, the present system is configured to obtain the NN intervals by applying a preprocessing step to filter out suspected ectopic beats, missed beats, and artifacts. In some embodiments, the present system may do so by analyzing the RR interval time series and discarding suspect intervals based on a criterion, e.g., an instantaneous increase by more than 20% in the RR interval with respect to surrounding RR intervals (Clifford, G. D., Behar, J., Li, Q., and Rezek, I. (2012). Signal quality indices and data fusion for determining clinical acceptability of electrocardiograms. Physiological Measurement 33, 1419-33. doi: 10.1088/0967-3334/33/9/1419).

In some embodiments, other known methods, such as range-based filtering; moving average filtering (see Mietus, J., and Goldberger, A. (2017). Heart Rate Variability Analysis with the HRV Toolkit—PhysioNet. Available from: https://physionet.org/tutorials/hrv-toolkit/); and/or quotient filtering (Piskorski, J., and Guzik, P. (2005). Filtering Poincaré plots. Computational Methods in Science and Technology 11, 39-48. doi:10.1016/j.jneumeth.2008.11.004) may be used. The particulars of each method and its adaptation to non-human mammalians are as follows:
Range-based filtering (RBF). RR interval duration inversely relates to the instantaneous heart rate:

$$IHR ( T ) = \frac{60}{R R ( t )}$$

where RR(t) is an RR interval, in seconds, measured at time t, and IHR(t) is the instantaneous heart rate in bpm at time t. This means that there are upper and lower bounds on the RR intervals that can be obtained from the physiological limits of the heart rate. For example, assuming that the instantaneous heart rate will be in the range of 40-187.5 bpm, then the range of RR interval values must be 0.32-1.5 seconds; intervals not within this range can thus be discarded as non-NN.
Moving average filter (MAF). In some cases, the RR interval time series can contain intervals that are very short or very long when compared to their neighboring intervals, and can appear as pronounced “spikes” when the RR intervals are plotted. While beat-to-beat variability is the desired result, these spikes indicate a significant change in RR interval duration between adjacent intervals or relative to neighboring interval values. This could, in turn, indicate that at least one of the beats in the interval was ectopic or perhaps even a false detection. To remove such intervals, a moving average filter is passed over the RR interval time series. The average in the window is calculated without the sample in the center of the window. If the central sample's value exceeds (in absolute terms) the window average by some percentage, that sample is removed. A window size of 21 samples may be used (10 samples on each side of the central sample) to filter out the sample if its value exceeded 20% of the window's average (default option corresponding to the ‘medium’ prefiltering level). The threshold and window size were chosen to be identical to the values used by the PhysioNet HRV toolkit (Mietus and Goldberger, 2017).
Quotient filter (QF). This filter removes intervals that vary by more than some percentage q from either the next or the previous interval (Piskorski and Guzik, 2005). It is conceptually similar to the sliding window average filter but much more local. For each interval, the filter examines the ratio between it and the previous and next intervals. If this ratio exceeds a q percent change, the interval is discarded. More specifically, there can be defined

$$r = \frac{q}{100} ( so 0 < r < 1 ) .$$

The filter rejects the interval at time tᵢ, RR(tᵢ), if at least one of the following conditions is true:

$$\frac{R R ( {t}_{i} )}{R R ( {t}_{i + 1} )} < 1 - r , or \frac{R R ( {t}_{i} )}{R R ( {t}_{i + 1} )} > 1 + r$$

$$\frac{R R ( {t}_{i + 1} )}{R R ( {t}_{i} )} < 1 - r , or \frac{R R ( {t}_{i + 1} )}{R R ( {t}_{i} )} > 1 + r$$

For humans, a value of q=20% was shown to work well for removing non-physiological intervals in data from heart-failure patients (Piskorski and Guzik, 2005). The same value (q=20%) may be used as a default option corresponding to the ‘medium’ prefiltering level. This filter is useful for removing strong local noise (very short or long intervals) due to its aggressiveness.

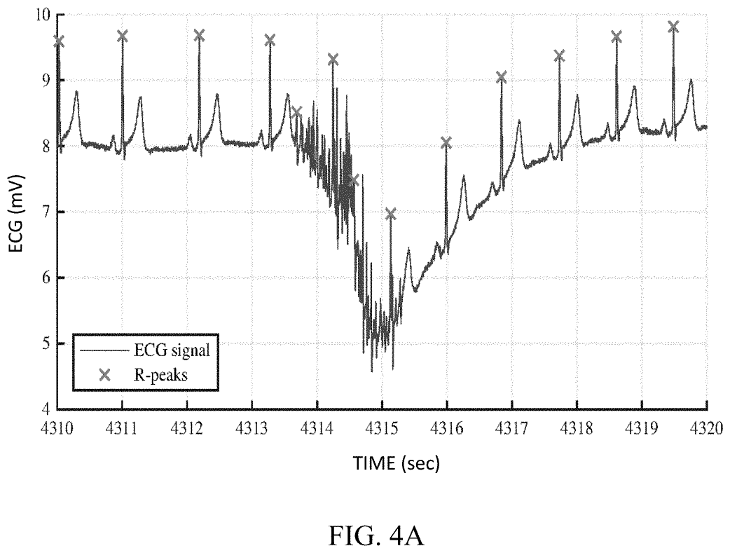

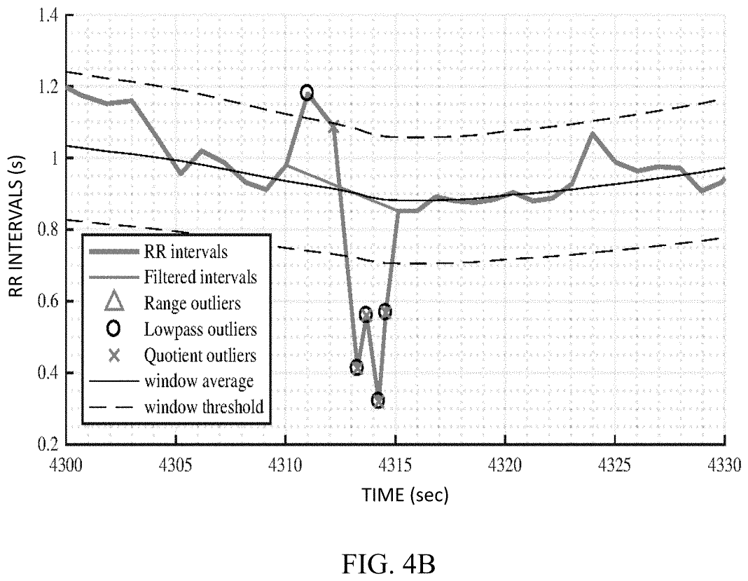

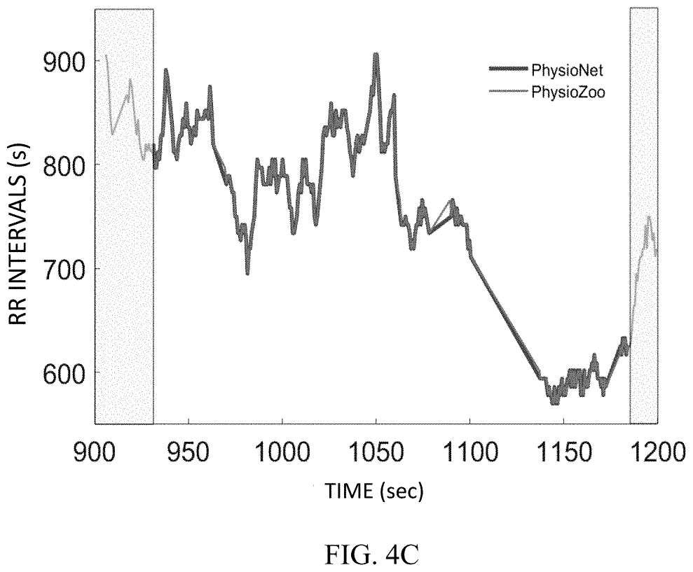

An example of noise leading to inaccurate R-peak detection is given in FIGS. 4A-4B. In FIG. 4A, there can be seen a relatively long interval starting at 4311 seconds, and a noisy region at 4313-4316 seconds. FIG. 4B shows RR interval time series before and after filtering, where the intervals are flagged for removal relative to the filter used. As can be seen, a series of four very short intervals start at 4313 seconds and correspond to a noisy region in the corresponding ECG (see FIG. 4A). In addition, there can be seen the quotient filter rejection of the interval at 4312 seconds, due to the large change relative to the next interval, as well as the moving average filter rejection of the interval at 4311 seconds due to it being more than 20% longer than the average in its neighborhood. FIG. 4C shows an example of the prefiltering step using the PhyioNet HRV toolkit, as well as the present system (termed PhysioNet) prefiltering stage on a 300 second ECG data signal. As can be seen, the PhysioNet HRV toolkit filter removes ˜40 seconds of the data due to the border effect, thus making the effective window closer to 260 seconds in length, which will in particular affect the minimal resolvable frequency

In some embodiments, following the prefiltering stage, the present system may then be configured to perform HRV detection with respect to the output data from the preprocessing stage. In some embodiments, the present system may be configured to employ one or more of power spectral density analysis, time domain analysis, frequency domain analysis, nonlinear analysis, detrended fluctuation analysis, sample entropy analysis, and fragmentation HRV analysis.

In some embodiments, the present system is configured for adapting one or more HRV parameters to various mammalian species. Because the HR range and dynamics can vary significantly between mammals, some HRV measures need to be adapted. For example, the pNNxx measure (see time domain analysis below) was adapted to dogs, rabbits, and mice. Similarly, several frequency-based measures were adapted to various mammal types. Table 3 below summarizes the HRV parameters selected for each species:

TABLE 3
Adaptation of HRV parameters in different mammals

| | Human | | | | |
|---|---|---|---|---|---|
| | (Task | | | | |
| | force)* | Human | Dog | Rabbit | Mouse |
| pNNxx threshold | 50 | 50 | 25 | 17 | 5 |
| (ms) | | | | | |
| VLF band (Hz) | 0.003-0.04 | 0.0033-0.046 | 0.0033-0.067 | 0.0033-0.088 | 0.0056-0.152 |
| LF band (Hz) | 0.04-0.15 | 0.046-0.158 | 0.067-0.235 | 0.088-0.341 | 0.152-1.240 |
| HF band (Hz) | 0.15-0.4 | 0.158-0.588 | 0.235-0.877 | 0.341-1.155 | 1.240-3.471 |
| Window size for | 5 | 5 | 5 | 5 | 3 |
| PSD analysis (min) | | | | | |

*See Malik, M. (Chairman of writing committee of task force of the European society of cardiology and the North American society of pacing electrophysiology) (1996). Heart Rate Variability: Standards of Measurement, Physiological Interpretation, and Clinical Use. Circulation 93, 1043-1065. doi:10.1161/01.CIR.93.5.1043

### Power Spectral Density Analysis

Power spectral density (PSD) analysis of the heartbeat intervals in three main frequency bands—very low frequency (VLF), low frequency (LF) and high frequency (HF)—provides a quantitative noninvasive tool for assessing the function of the cardiovascular control system. In humans, these frequency bands were standardized following years of empirical evidence. In healthy humans, the frequency of the PSD performed on a 5-minute long recording is traditionally divided into the HF band, 0.15 to 0.4 Hz, where the dominant HF peak can typically be found around 0.25-0.3 Hz; the LF band, 0.04 to 0.15 Hz, where the dominant peak can typically be found around 0.1 Hz; and the VLF band, 0.0033-0.04 Hz. The HF band corresponds to rhythms with periods between 2.5 and 7 seconds and is known to reflect parasympathetic (vagal) activity and respiratory sinus arrhythmia. The LF band corresponds to rhythm modulations with periods between 7 and 25 seconds and is believed to mainly reflect baroreflex activity while at rest. The LF was also often assumed to have a dominant sympathetic component, but this assumption has been strongly challenged. The VLF corresponds to rhythms with periods between 25 and 300 seconds. VLF power has been strongly associated with all-cause mortality in cohorts with cardiac failure or multiple organ dysfunction syndrome. The energy contained in this band has been suggested to be intrinsically generated by the heart. However, no quantitative approach has justified the frequency cutoffs of these bands and how they might be adapted to non-human mammals. Accordingly, by defining mammal-specific frequency bands, PSD analysis of HRV is to be used as a proxy for measuring the autonomic nervous system activity in animal models.

Accordingly, in some embodiments, the present invention provides for adapting PSD analysis to non-human mammals. In some embodiments, the present invention begins by deriving the distribution of prominent frequency peaks found in the normalized PSD of mammalian data using a Gaussian mixture model, while assuming three components corresponding to the traditional VLF, LF and HF bands. For example, an algorithm may be trained on a large database of human electrocardiogram recordings (n=18), and then validated on databases of dogs (n=17) and mice (n=7). The algorithm may then be tested to predict the bands for rabbits (n=4) for the first time. Double-logarithmic analysis demonstrates a scale law between the GMM-identified cutoff frequencies and the typical heart rate (HR.):

f_{VLF-LF}=0.0037·HRₘ^{0.58},f_{LF-HF}=0.0017·HRₘ^{1.01}

and

f_{HF}_{ᵤₚ}=0.0128·HRₘ^{0.86}.

Accordingly, it was found that the band cutoff frequencies and Gaussian mean scale with a power-law of ¼ or ⅛ of the typical body mass (BMₘ), thus revealing allometric power laws. Thus, in some embodiments, the present invention is based on the finding that the scaling law between the band frequency cutoffs and the HRₘ can be used to approximate the PSD bands in other mammals.

In some embodiments, the empirical data from which the scaling law finding is derived are based, at least in part, on ECG recordings from healthy humans (n=18), heartworm-free mixed-breed dogs (n=17), C57BL/6 male mice (n=7), and New Zealand white rabbits (n=4). These data were then preprocessed to detect RR and NN intervals.

In PSD, the upper bound of the HF band (f_{HF}_{ᵤₚ}) defines the minimal resampling frequency of the NN time series (i.e., fₛ≥2·f_{HF}_{ᵤₚ}, following Shannon's criterion). In the literature, f_{HF}_{ᵤₚ} was set to 0.4 Hz in humans, but there is no clear rationale for this cutoff. Accordingly, the PSD was computed up to the maximal frequency meaningful to resolve (fₘₐₓ), which was set as the frequency corresponding to the ‘characteristic shortest RR interval’ found in normal sinus rhythm electrocardiogram recordings. The ‘characteristic shortest RR interval’ was then defined as the lower bound of the interval containing 95% of the RR intervals from a large population. Given fₘₐₓ, the PSD was estimated on the interval [0 fₘₐₓ] Hz using the Welch periodogram (Welch, 1967). Because Welch PSD estimation requires uniformly-sample data, the RR interval time series was resampled at 2.2 times fₘₐₓ. The resampling frequency was chosen such that it satisfies Shannon's criterion.

To perform PSD analysis, a window (i.e., sub-segment) of the NN time series must be selected. This window must be long enough to resolve the VLF band and short enough to assume data stationarity. The traditional window for humans is 5 minute long. Because no alternative window length has been standardized for dogs and rabbits, the same window size was used as for humans (i.e., 5 minute). A 3 minute window was used for mice, as suggested in Thireau et al. (Thireau et al., 2008). The frequency band starting from 0.0033 Hz for humans, dogs and rabbits is denoted as ‘VLF,’ (corresponding to the lowest frequency which can be resolves using a 5 min window size 1/(60*5)), and 0.0056 Hz for mice (corresponding to the lowest frequency which can be resolved using a 3 min window size 1/(60*3)), up to the upper bound of the VLF band. In summary, non-overlapping 5 minute windows were used for human, dog and rabbit data and non-overlapping 3 minute windows were used for the mouse data. PSD was computed for each non-overlapping window. In the instances where the recording was less than the window size length, the PSD was computed on the total recording length available. This happened in particular for the dog data, where a number of recordings were 4-5 minutes long.

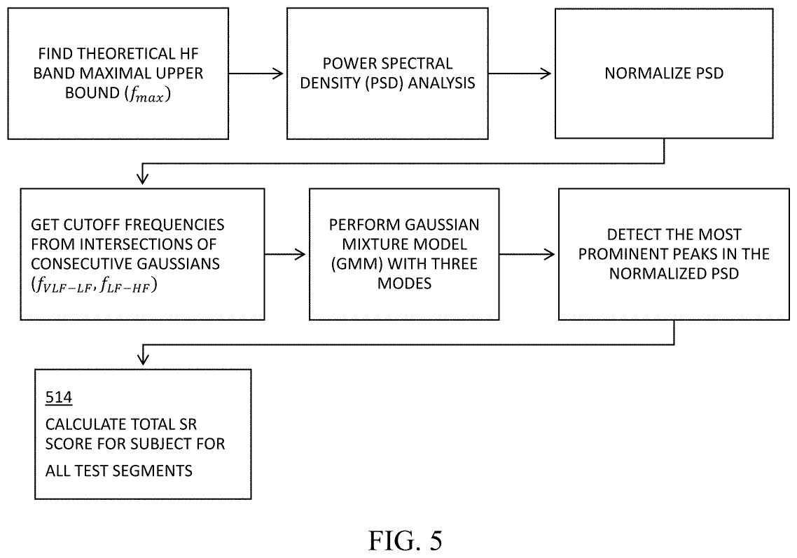

The PSD was then normalized for each non-overlapping window by the total power in order to allow cross-comparison of mammal types. For each normalized PSD there were detected prominent frequency peaks. Given the location of the detected peaks for each window size, a histogram of the prominent peak locations was created for each mammal type. It was further assumed that the histograms were generated from a mixture of Gaussian distributions; thus, a Gaussian Mixture Model (GMM) was used to estimate the Gaussian parameters that best describe the underlying distribution. Because of the three traditional power spectral bands (VLF, LF and HF), three Gaussians were used for the GMM (thus assuming three modes). The intersection between consecutive Gaussians was defined as the band cutoff frequencies. To set the minimal peak height threshold (defined as the minimal amplitude a peak must have in the normalized PSD to be considered as ‘prominent’), the human data was used. Given the selected minimal peak height threshold for humans, the same algorithm was applied to the dog, rabbit and mouse data. The upper bound of the HF (f_{HF}_{ᵤₚ}) band was defined as three standard deviations away from the mean of the Gaussian describing the HF band, thus covering 99.7% of the prominent peak density in the HF band. Note that fₘₐₓ defined the upper frequency bound up to which the PSD is computed, whereas f_{HF}_{ᵤₚ} corresponds to the final upper cutoff frequency of the HF band as estimated from the GMM approach. A block diagram summarizing the main steps to identify the frequency bands is shown in FIG. 5. The algorithm was then trained on a database of human electrocardiogram recordings (n=18), and validated on databases of dogs (n=17) and mice (n=7). Finally, the algorithm was tested to predict the bands for rabbits (n=4) for the first time.

Given the bands identified by the GMM for humans, dogs, rabbits and mice, a power-law relationship was derived between the band cutoff frequencies or mean of each Gaussian and HRₘ. HRₘ was defined as the median HR evaluated on the database for a given mammal. To search for power-laws between band cutoff frequencies and HRₘ, a double-logarithmic analysis of the band cutoffs was used for each mammal type against HRₘ. To search for a power-law between the dominant peak in each of the GMM-identified band and HRₘ, a double-logarithmic analysis of the dominant peak location for each 5- or 3-minute segment against the mean HR of the segment was used. In addition, a allometric power law was searched as between the band cutoff frequencies and the BMₘ. For that purpose, a double-logarithmic analysis of the band cutoffs was used for each mammal type against the typical BMₘ of the mammals included in the database. A linear regression was used to explore whether a linear relationship existed between the variables in the double-logarithmic plot.

Accordingly, the human ECG data was used to train a GMM. The median RR interval for humans was 766 ms and the lower bound of the RR distribution (thus describing the ‘fastest’ heart rate) was 359 ms. Therefore, fₘₐₓ was set to 2.8 Hz. Based on GMM fitting, the VLF band was identified to be between 0.0033 and 0.046 Hz, the LF band between 0.046 and 0.158 Hz, and the HF band between 0.158 and 0.588 Hz.

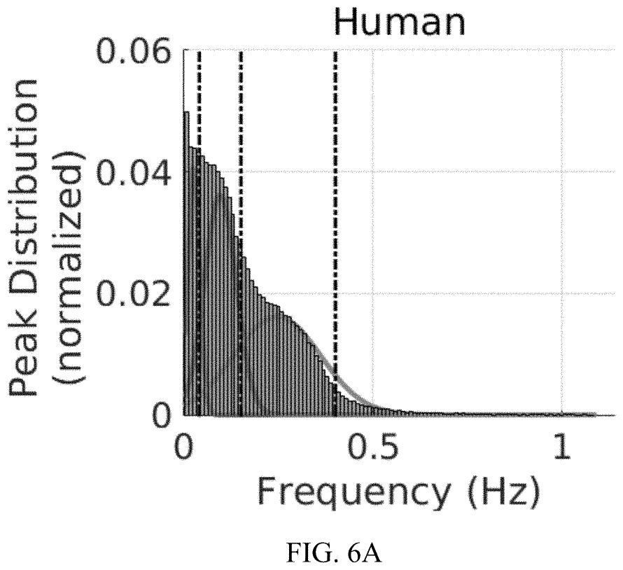

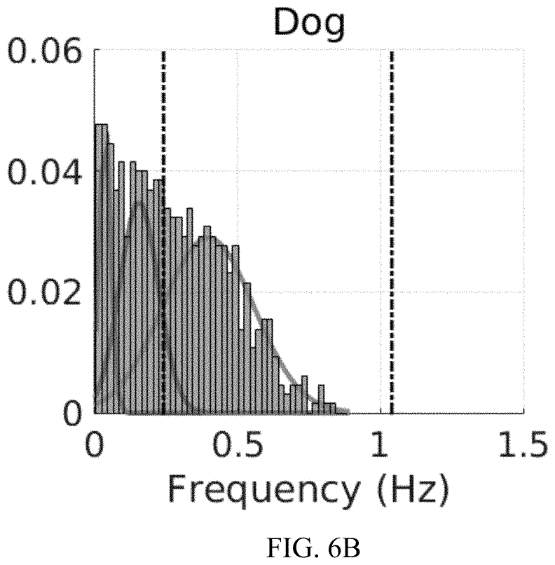

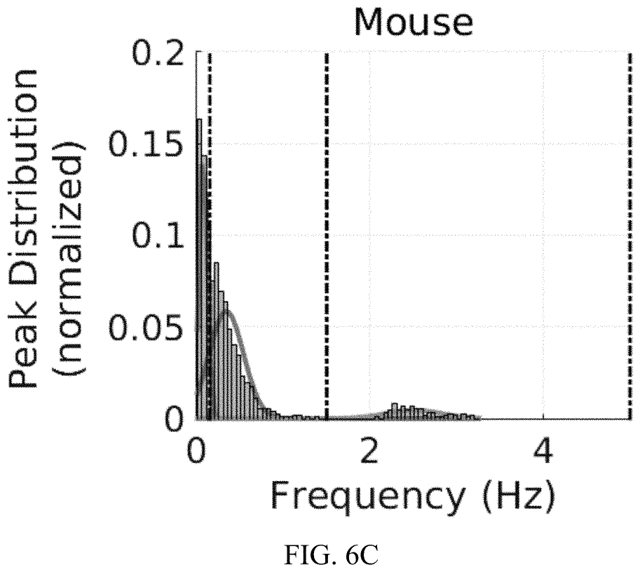

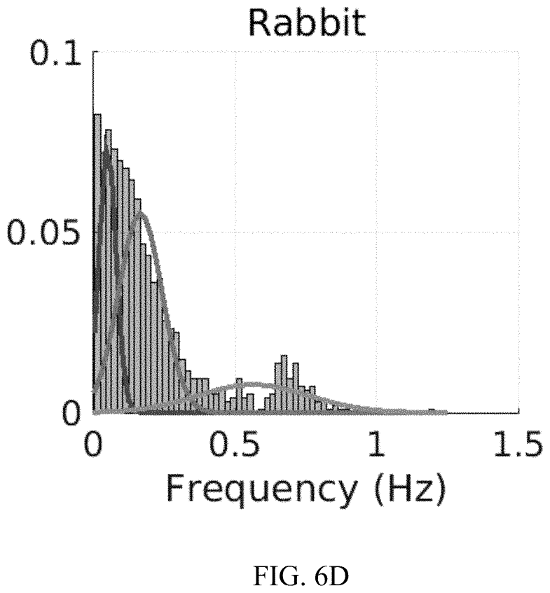

FIGS. 6A-6D show Gaussian mixture models estimated for various mammals. The results were validated on data from dogs and mice data. The median RR interval for dogs was 484 ms and the 97.5% lower bound of the RR distribution was 360 ms. Thus fₘₐₓ was 2.8 Hz. Using the GMM model trained on the human data, the bands were estimated for the dog data; the VLF band was found to be between 0.0033 and 0.067 Hz, the LF band between 0.067 and 0.235 Hz, and the HF band between 0.235 and 0.877 Hz. Using the GMM model trained on the human data, the bands were estimated for the mice data; the VLF band was identified to be between 0.0056 and 0.152 Hz, the LF band between 0.152 and 1.240 Hz, and the HF band between 1.240 and 3.471 Hz. Using the GMM approach trained on the human data, the bands were also estimated for the rabbit data; the VLF band was identified to be between 0.0033 and 0.088 Hz, the LF band between 0.088 and 0.341 Hz, and the HF band between 0.341 and 1.155 Hz.

In addition to the frequency band cutoffs, the Gaussian means and standard deviations may also be reported for each band of each mammal (see table 4 below).

TABLE 4
Gaussian mean (μ) and standard deviations (σ)
(numbers are rounded to the third decimal place)

| Band | Human (μ ± σ) | Dog (μ ± σ) | Rabbit (μ ± σ) | Mouse (μ ± σ) |
|---|---|---|---|---|
| G_{VLF} | 0.026 ± 0.015 | 0.035 ± 0.020 | 0.049 ± 0.030 | 0.074 ± 0.046 |
| G_{LF} | 0.095 ± 0.042 | 0.154 ± 0.068 | 0.165 ± 0.077 | 0.339 ± 0.198 |
| G_{HF} | 0.246 ± 0.114 | 0.397 ± 0.160 | 0.564 ± 0.197 | 2.505 ± 0.322 |

Finally, the power ratio was computed: the relative power of each band over the total power for each mammal (see table 5 below). The data show that the HF power is relatively higher in larger mammals (humans and dogs) than in smaller mammals (rabbits and mice).

TABLE 5
Power ratio (of relative power over total power, %)
for the different bands.

| | Human | Dog | Rabbit | Mouse |
|---|---|---|---|---|
| VLF (%) | 55.5 | 40.0 | 71.5 | 63.8 |
| LF (%) | 28.0 | 20.6 | 19.4 | 25.8 |
| HF (%) | 19.0 | 40.7 | 10.4 | 11.0 |

Based on the above, a power ratio was computed, based on the relative power of each band over the total power for each mammal. First, the GMM approach was used for defining the frequency bands to determine whether a universal scaling relation exists between HRₘ and the PSD band cutoff frequencies between VLF and LF (f_{VLF-LF}) or LF and HF (f_{LF-HF}) or the upper bound frequency of the HF band (f_{HF}_{ᵤₚ}). By examining the median HRₘ for humans and mice in our database, a median HRₘ was obtained that ranges from 78 to 550 bpm. Similarly, f_{VLF-LF} ranges from 0.046 to 0.152 Hz, f_{LF-HF} ranges from 0.158 to 1.240 Hz and f_{HF}_{ᵤₚ} from 0.588 to 3.471 Hz.

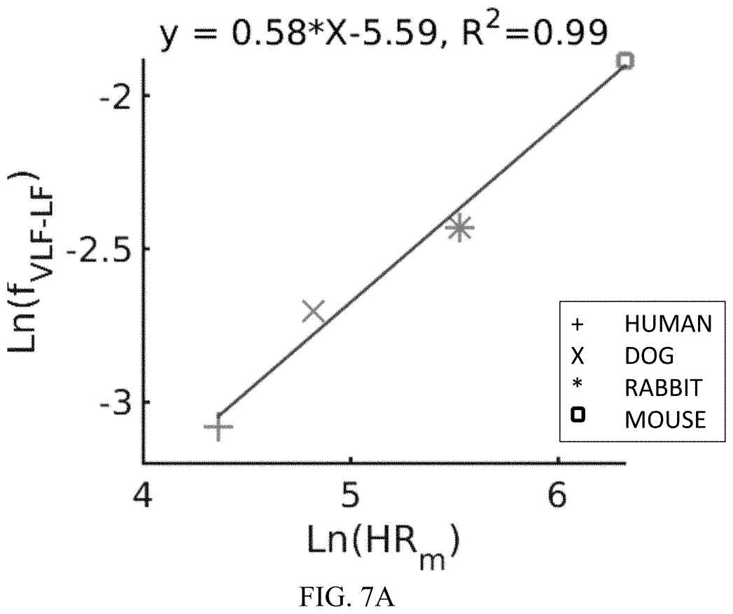

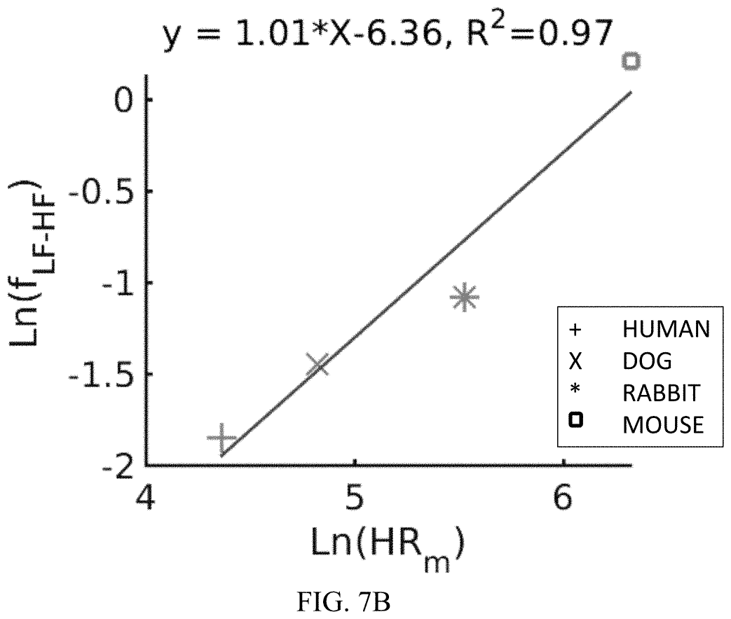

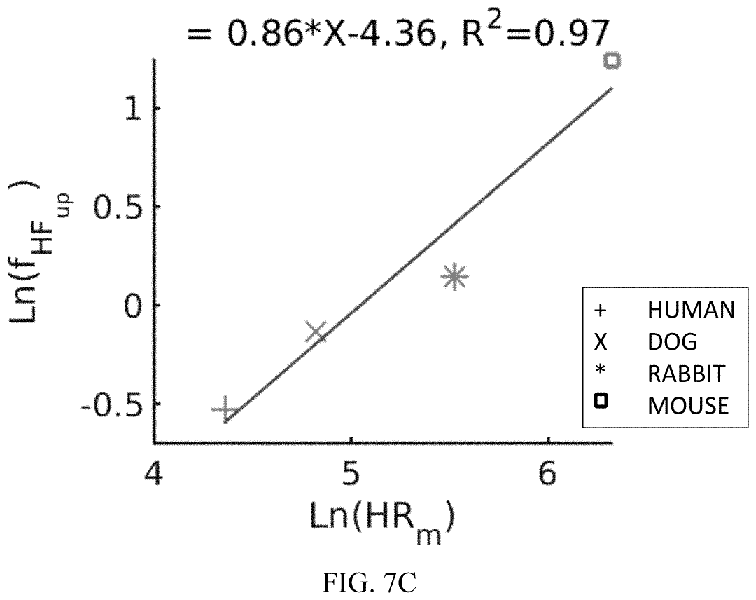

In FIG. 7A, the ln(f_{VLF-LF}) is plotted against ln(HRₘ). The cutoff frequency scales with the HRₘ following the power-law: f_{VLF-LF}=0.0037·HRₘ^{0.58}. In FIG. 7B, the ln(f_{LF-HF}) was plotted versus ln(HRₘ). Thus, f_{LF-HF} scales with HRₘ as f_{LF-HF}=0.0017·HRₘ^{1.01}. In FIG. 7C, the ln(f_{HF}_{ᵤₚ}) is plotted versus ln(HRₘ), whose best linear fit is y=0.86·x−4.36. Thus, f_{HF}_{ᵤₚ} scales with HRₘ following the power-law: f_{HF}_{ᵤₚ}=0.0128·HRₘ^{0.86}.

Second, the GMM approach was used to determine whether a universal scaling relation exists between HRₘ and the mean of the Gaussian describing each band.). From the linear fit, the following power-law relationships were found: G_{VLF}=0.0027·HRₘ^{0.53}, G_{LF}=0.0077·HRₘ^{0.59} and G_{HF}=0.0016·HRₘ^{1.13}.

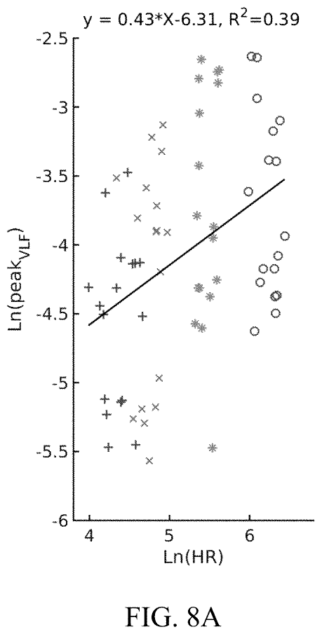

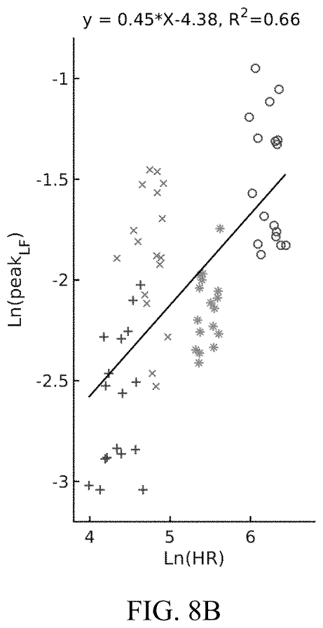

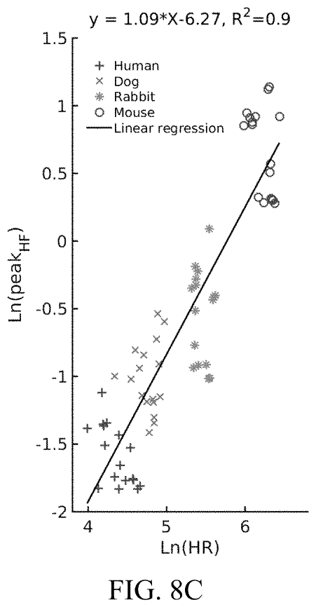

Third, there was determined whether a universal scaling relation exists between the HR and the dominant PSD peak in each of the frequency bands. In FIGS. 8A-8C, ln(peak_{band}) is plotted for each frequency band (VLF, LF and HF) versus ln(HR), respectively. The data indicate that only the peak_{HF} is correlated with the HR. The dominant peak in the HF band scales with HR as peak_{HF}=0.0019·HR^{1.09}.

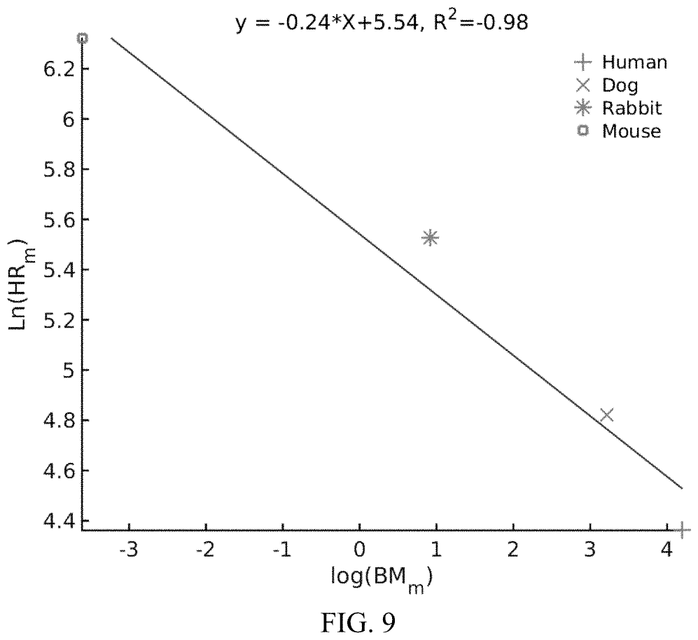

Fourth, there was determined whether a universal law for allometric scaling in biology also exists between BMₘ and the band cutoff frequencies identified by the GMM. It was first checked that a −¼ allometric law between HRₘ and BMₘ could be retrieved form the data. FIG. 9 illustrates the result of the line fit for this analysis. The scaling power found on our data was −0.24, which is very close to −¼, as expected. From the linear fit, the following power-law relationships were found: f_{VLF-LF}=0.0944·BMₘ^{-0.14}, f_{LF-HF}=0.4771·BMₘ^{-0.26} and f_{HF}_{ᵤₚ}=0.644·BMₘ^{-0.22}.

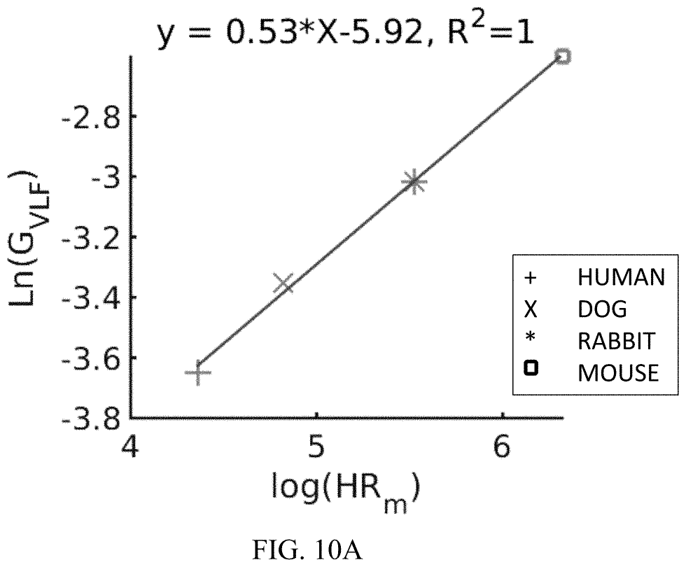

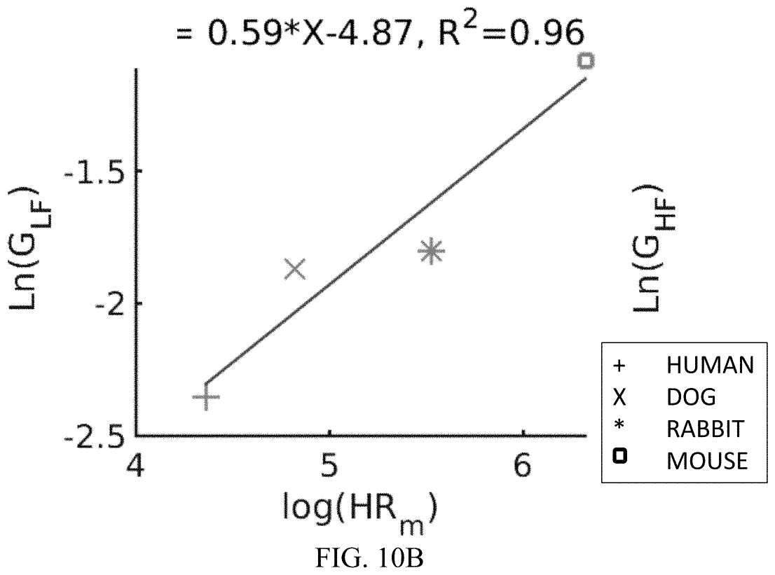

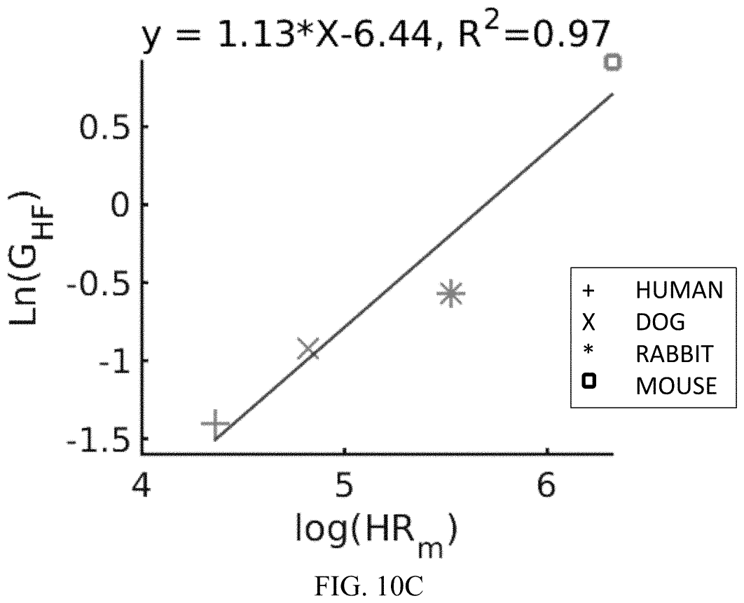

Finally, it was determined whether a universal law for allometric scaling in biology also exists between BMₘ and the mean of the Gaussians describing each band. In FIG. 10A, G_{VLF} was plotted against ln(BMₘ and an allometric law of G_{VLF}=0.0493·BMₘ^{-0.13} was found. In FIG. 10B, ln(G_{LF}) was plotted against ln(BMₘ), and an allometric law of ln(G_{LF})=0.1999·BMₘ^{-0.14} was found. In FIG. 10C, ln(G_{HF}) was plotted against ln(BMₘ), and an allometric law of ln(G_{HF})=0.8521·BMₘ^{-0.29} was found.

In some embodiments, the PSD analysis described above provides for identifying the frequency bands in PSD analysis of heartbeat interval time series from different mammals. For the human data, the disclosed PSD analysis identified the bands as: VLF [0.0033-0.046] Hz, LF [0.046-0.158] and HF [0.158-0.588] Hz. These results are comparable to the recommended bands known in the literature (VLF [0.0033-0.04], LF [0.04-0.15] and HF [0.15-0.4]).

In some embodiments, the PSD analysis described above provides for the application of the PSD method to dog, rabbit and mouse ECG data. Using ECG data from healthy and awake animals, the frequency cutoffs between the VLF-LF and LF-HF bands could be defined.

This model was validated by comparing its results to bands experimentally identified in the literature for dogs and mice: the cutoff between LF and HF in dogs data is similar to the one used in the work of Billman et al. (Billman, 2013a). Similar cutoffs are also documented for mice: the cutoff between VLF and LF and between LF and HF is similar to other works (Adachi et al., 2006; Baudrie et al., 2007; Campen, 2005; Farah et al., 2006; Fazan, 2005; Ishii et al., 1996; Joaquim, 2004; Just et al., 2000; Tankersley et al., 2004; Thireau et al., 2008). Finally, the model was tested on the rabbit dataset. One study (Goldstein et al., 1995) has looked into defining frequency bands in rabbits. However, this study was performed on anesthetized rabbits, which may require different band definitions than the ones for conscious rabbits, particularly because of the changes in HR that can be caused by the different agents used and the depth of anesthesia (Mazzeo et al., 2011; Picker et al., 2001). Thus, the HRV frequency bands for this mammal were defined for the first time. The prominent peak found in the HF band is characteristic of the respiratory rate (respiratory sinus arrhythmia) (Akselrod et al., 1981). Therefore, the mean of the Gaussian describing the HF band is expected to fall within the respiratory range of the corresponding mammal. The values identified by the GMM (0.397, 0.564 and 2.505 Hz for dogs, rabbits and mice, respectively) fall within the respiratory rate range for these three mammals: 20-40 breaths per minute (brpm) for dogs [0.33-0.67] Hz, 30-60 brpm for rabbits [0.5-1] Hz, and 60-220 brpm for mice [1-3.67] Hz (University Wisconsin-Madison, 2018). Thus the GMM approach successfully retrieved the vagal activity enabled by respiratory effort and which is known to manifest in the HF band.

In some embodiments, the PSD analysis disclosed above provides for the definition of the upper bound of the HF bands. The upper bound of the HF band is defined as being three standard deviations away from the mean of the Gaussian representing the HF band. The upper boundary of the HF band for humans was 0.588 Hz. Using the same method, the cutoff frequencies defined for the other mammals were 0.877, 1.155 and 3.471 Hz for dogs, rabbits, and mice, respectively. Interestingly, the present results show that the mouse may serve as a better mammalian model than do the dog or rabbit for studying the effects of drugs, mutations, or cardiac diseases on vagal activity as reflected in the HF band. Indeed, the Gaussian fittings for mice show minimal overlap between the LF and HF bands compared to other mammals. This means that the vagal activity in mice can be studied without interference from the physiological processes echoed in the low frequency band. However, this is moderated by the larger HF power ratio for humans and dogs (19 and 40.7%, respectively) versus rabbits and mice (10.4 and 11%, respectively). This means that rabbits and mice display relatively less vagal activity than humans and dogs. The effect of respiration is dominant in the HF band. However, during periods of slow respiration, the resulting vagal activity will modulate the HR at frequencies which will cross over into the LF band. Thus, for a breathing rate lower than 9.6 brpm, the characteristic vagal frequency peak will fall in the LF band in humans. This observation may be interpreted as being due to the higher breathing rates of smaller mammals, which leads to a better separation between the respiratory sinus arrhythmia modulation of the heart rate (reflected mainly in the HF band) and other autonomic nervous system effects, partly reflected in the LF bands.

In some embodiments, the PSD analysis disclosed above also provides for a power-law relationship between band cutoff frequencies, dominant PSD peak, and HRₘ. Thus, a relationship exists between our GMM-identified cutoff frequencies and HRₘ. This scaling law can be used to approximate the PSD bands for other mammals not included in in the present study. In order to define the bands for nonhuman mammals, other works have scaled the frequency bands used in humans linearly with the ratio of human HRₘ and HRₘ of another mammal. However, this approach is incorrect because the relationship between the frequency cutoffs and the HRₘ is not linear with respect to the ratio of the mean heart rates, as the equations for the log-log subplots highlight. Interestingly, a scaling relation could only be found between the dominant HF peak and HR. Because the HF peak represents vagal activity and because the scale power was 1, the present results suggest that vagal activity in different mammals is linearly associated with HR. The prominent peak found in the HF band shifts with changes in the respiratory rate, and thus a scaling law was shown between the respiratory rate and the typical HR across mammals. This was tested as to whether the power law can be used to approximate the HRV band cutoff frequencies from other species than the ones included in this work. Aubert et al. (Aubert et al., 1999) have investigated frequency bands in experimental rat models using direct manipulation of autonomic parameters through pharmacological intervention. They identified the LF and HF bands as: LF [0.19-0.74] and HF [0.78-2.5] Hz. The mean HR of the rat population used by Aubert et al. (Aubert et al., 1999) was 345 bpm. Using this typical HR value and the power law established between HRₘ and the PSD cut-off frequencies, the bands were predicted as: VLF [0.0033-0.110] Hz, LF [0.110-0.622] and HF [0.622-1.949] Hz, which is close to the experimental findings of Aubert et al. (Aubert et al., 1999).

In some embodiments, the PSD analysis disclosed above also provides for the finding that the band cutoff frequencies in mammals, f_{LF-HF} and f_{HF}_{ᵤₚ}, follow an allometric law that scales as the ˜−¼ power of the body mass and that G_{VLF} and G_{LF} scale as the ˜−⅛ power of the body mass. Interestingly, the ¼ scaling power has been shown previously to be an essential component in biological scaling (West et al., 1997). In the model of West et al. (West et al., 1999), processes that rely on hierarchical networks for resource distribution are identified to scale with BMₘ^{n/4} (with n∈ [symbol] ). In practice, many variables in mammalian physiology have been shown to scale with BMₘ following such a quarter-power law. These include metabolic rate, circulation time, HR, aortic diameter, and respiratory rate (Noujaim et al., 2004; West et al., 1997). Because the frequency bands correspond to the regulation of specific physiological processes (such as baroreflex or vagal activity) on the HR dynamic, it was interesting to find that the band cutoff frequencies(f_{LF-HF}, f_{HF}_{ᵤₚ}) and the BMₘ also followed this universal quarter allometric scaling to ensure optimal autonomic activity in regulating the HR dynamics.

### Time Domain Analysis

The most straightforward HRV analysis methods rely on linear time-domain statistics. For i=0, . . . , N−1, tᵢ will be denoted as the beat timestamp and and NN(tᵢ) as the NN interval beginning at time tᵢ. The following well-known measures were implemented.
SDNN: The standard deviation of all NN intervals.

$$S D N N = \sqrt{\frac{1}{N - 1} {\sum }_{i = 0}^{N - 1} {( N N ( {t}_{i} ) - \overline{NN} )}^{2}} \qquad ( 5 )$$

where N̅N̅ is the mean NN interval time.
RMSSD: Root mean square of successive differences (SD) of NN intervals.

$$RMSSD = \sqrt{\frac{1}{N - 1} {\sum }_{i = 0}^{N - 1} {( N N ( {t}_{i} ) - N N ( {t}_{i - 1} ) )}^{2}} . \qquad ( 6 )$$

pNN50: The percentage of NN intervals which differ by at least 50 ms from their preceding interval:

$$p N N 50 = \frac{1}{N - 1} {\sum }_{i = 1}^{N - 1} {I}_{i}^{50} \qquad ( 7 )$$

where lᵢ⁵⁰ is the indicator

$${I}_{i}^{50} = \{ \begin{matrix}1 , & \vert N N ( {t}_{i} ) - N N ( {t}_{i - 1} ) \vert > 50 [ ms ] \\ 0 , & else\end{matrix} .$$

In particular, the pNN50 measure was derived from the original study of Ewing et al. (Ewing et al., 1984). The authors introduced the NN50 measure, defined as the mean number of time per hour in which the change in the NN intervals exceeded 50 ms. The authors suggested this measure to assess parasympathetic (vagal) activity from 24-hour ECG recordings. Later, Bigger et al. introduced the pNN50 measure. The pNN50 is a member of the broader class of statistics, pNNxx. A study on optimizing the xx value was conducted by Mietus et al..

In some embodiments, the pNNxx measure may use a fixed criterion for variability, based on a threshold that was found to work well for human ECG data (xx=50 ms) to quantify vagal activity. This threshold needs to be adapted for different mammals. Assuming that the respiratory rate frequency is characteristic of the vagal activity contribution to HRV, the ratio between the average breathing cycle length of the corresponding mammal was divided by the average breathing cycle length of humans (rounded value). The respiratory rate range for humans is 12-18 breaths per minute (brpm), 20-40 brpm for dogs, 30-60 brpm for rabbits, and 60-220 brpm for mice. This led to values of 25 ms, 17 ms and 5 ms for dogs, rabbits, and mice, respectively.

### Frequency Domain Analysis

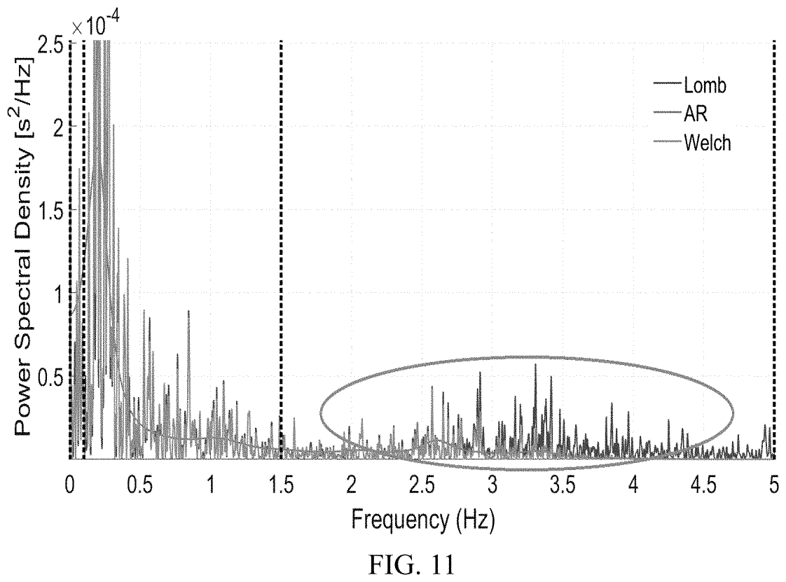

A number of methods exist for spectral estimation. These methods can be broadly categorized as parametric and nonparametric. Parametric methods rely on a predetermined model of the process producing the samples, usually an autoregressive (AR) moving average model. Nonparametric methods, typically the Welch (Welch, 1967) or Lomb methods (Lomb, 1976), compute the spectrum directly usually using Fourier-based analysis of the data itself. In particular, many cardiologists prefer to use the AR model because it is easier to visually identify the characteristic LF and HF peaks with this ‘smoother’ representation versus the nonparametric methods. While the Lomb method eliminates the need for resampling and thus prevents the artifacts associated with it, the risk of aliasing (see FIG. 11) is higher with this method because the maximal frequency which can be resolve without aliasing greatly depends on the average HR in the recording. For this reason, in some embodiments, the present system only enables the AR and Welch methods for PSD analysis (see discussion of PSD above).

### Nonlinear Measures

The fractal methods require no adaptation. However, when using a power spectral density (PSD) based estimate of the β coefficient (slope of the linear interpolation of the spectrum in a log-log scale for frequencies below the upper bound of the VLF band), the appropriate VLF frequency band must be used for the corresponding mammal. Entropy methods do not require adaptation for different mammals.

### Experimental Results

The MIT normal sinus rhythm database (Goldberger, A. L., Amaral, L. A. N., Glass, L., Hausdorff, J. M., Ivanov, P. C., Mark, R. G., et al. (2000b). PhysioBank, PhysioToolkit, and PhysioNet. Circulation 101. doi:10.1161/01.CIR.101.23.e215) was used to benchmark between the HRV measures from the present system and the PhysioNet HRV Toolkit. The first 24 hours of each file in the NSR database were taken into account to make the comparison. Table 6 below reports the mean (μ) and standard deviation (a) of the time HRV measures over the NSR database using the two tools, as well as the root mean square error (RMSE) and the normalized root mean square error (NRMSE) of the comparison between the HRV Toolkit and the present system. The NRMSE was defined as being the RMSE normalized by the mean of the given HRV measures computed using the HRV Toolkit (used as the reference).

As can be seen in table 6 below, the time domain HRV measures gave similar results to the PhysioNet HRV Toolkit when benchmarked on the MIT normal sinus rhythm database. The normalized root mean square error was small for all benchmarked measures. The residual differences may be attributed to to the following: (1) the PhysioNet HRV Toolkit removes data points on the edge of the windows; (2) the prefiltering step is slightly different in our implementation versus the PhysioNet HRV toolkit; and (3) errors due to the difference in rounding. However, the errors are minor and should not affect HRV analysis.

TABLE 6
Validation of some standard HRV measures

| | HRV Toolkit | | Present System | | Error | |
|---|---|---|---|---|---|---|
| | μ | σ | μ | σ | RMSE | NRMSE |
| NN | 0.9781 | 0.0233 | 0.987 | 0.0137 | 0.0131 | 0.0133 |
| AVNN | 800.97 | 84.741 | 802.1 | 84.703 | 2.3112 | 0.0029 |
| SDNN | 131.02 | 41.068 | 131.84 | 41.137 | 1.619 | 0.0124 |
| RMSSD | 37.652 | 14.335 | 39.342 | 15.874 | 2.9266 | 0.0777 |
| pNN50 | 14.124 | 9.6752 | 14.726 | 10.097 | 1.0453 | 0.0740 |

Table 7 below summarizes the HRV measures implemented in the present system software for dogs, rabbits, and mice:

TABLE 7
Summary of HRV measures

| | | |
|---|---|---|
| Measures | Units | Definition |

Time domain

| | | |
|---|---|---|
| AVNN | [ms] | Average NN interval duration |
| SDNN | [ms] | Standard deviation of NN interval duration |
| RMSSD | [ms] | The square root of the mean of the sum of the squares |
| | | of differences between adjacent NN intervals |
| pNNxx | [%] | Percent of NN interval differences greater than xx |
| | | milliseconds |
| SEM | [ms] | Standard error of the mean NN interval |
| PIP | [%] | Percentage of inflection points in the NN |
| | | interval time series |
| IALS | [n.u] | Inverse average length of the acceleration/ |
| | | deceleration segments |
| PSS | [%] | Percentage of short segments |
| PAS | [%] | The percentage of NN intervals in alternation segments |

Frequency

| | | |
|---|---|---|
| Total | [ms²] | Total power |
| power | | |
| VLF | [ms²] | Power in the very low frequency band |
| LF | [ms²] | Power in the low frequency band |
| HF | [ms²] | Power in the high frequency band |
| LF norm | [%] | Low frequency power in normalized units LF/ |
| | | (Total power − VLF) × 100 |
| HF norm | [%] | High frequency power in normalized units: HF/ |
| | | (Total power − VLF) × 100 |
| LF/HF | [n.u] | Low frequency band to high frequency band |
| | | power ratio (LF/HF) |
| LF peak | [Hz] | Peak frequency in the low frequency band |
| HF peak | [Hz] | Peak frequency in the high frequency band |

Nonlinear

| | | |
|---|---|---|
| β | [n.u] | Slope of the linear interpolation of the spectrum in a |
| | | log-log scale for frequencies below the upper bound |
| | | of the VLF band |
| SampEn | [n.u] | Sample entropy |
| SD1 | [ms] | NN interval standard deviation along the perpendicular |
| | | to the line-of-identity |
| SD2 | [ms] | NN interval standard deviation along the |
| | | line-of-identity |
| α₁ | [n.u] | DFA low-scale slope |
| α₂ | [n.u] | DFA high-scale slope |

All HRV parameters in the present system can be manually changed by the user by editing a single configuration file. This configuration file may also be uploaded as a supplement to a publication in order to support the reproducibility of the results, as it contains all the parameters required to reproduce the analysis.

Table 8 below summarizes the mean values (±STD) for all the HRV measures implemented in the present system for all mammals. This provides a standard reference HRV range obtained from conscious and healthy mammals at rest. Mean (μ) and standard deviation (σ) of the HRV measures for the mammal databases included in the present system and from the PhysioNet normal sinus rhythm database for humans. The human, rabbit and mouse databases were preprocessed with moderate combined filtering (i.e., range plus moving average filter), and the dog database was preprocessed with the combined filtering with a moving average filter threshold at 40%. Thus, for example, using the mean SampEn measures computed in table 8 for each mammal, the power law relationship between the mean SampEn and HRₘ or BM of the different mammals can be found. The results suggest that the complexity (in the sense expressed by sample entropy) of the heart rate increases with HRₘ and decreases with BM. This in turn suggests that the complexity of the heart rate decreases in smaller mammals.

TABLE 8
Mean values for all HRV measures.

| | Human* | Dog | Rabbit | Mouse |
|---|---|---|---|---|
| | (p = 4013, n = 18) | (p = 17, n = 17) | (p = 34, n = 4) | (p = 65, n = 7) |
| | μ ± σ | μ ± σ | μ ± σ | μ ± σ |
| AVNN (ms) | 798 (±1.80e4) | 520 (±8.36e3) | 255 (±779) | 115 (±264) |
| SDNN (ms) | 59 (±761) | 75.2 (±1.11e3) | 10.1 (±20.9) | 10.5 (±32.1) |
| RMSSD (ms) | 37.3 (±522) | 75 (±2.87e3) | 4.39 (±4.27) | 5.37 (±14.1) |
| pNNxx (%) | 14.9 (±278) | 53.7 (±415) | 0.789 (±1.53) | 19.2 (±344) |
| SEM (ms) | 3.12 (±2.8) | 3.38 (±3.54) | 0.299 (±2.07e−2) | 0.272 (±2.49e−2) |
| PIP (%) | 40.5 (±68.1) | 47.4 (±85.6) | 43.8 (±36.2) | 44.2 (±105) |
| IALS (n.u.) | 0.409 (±6.91e−3) | 0.476 (±8.99e−3) | 0.44 (±3.69e−3) | 0.443 (±1.05e−2) |
| PSS (%) | 39.9 (±292) | 48.8 (±303) | 38.4 (±82) | 41.5 (±249) |
| PAS (%) | 6.98 (±40.3) | 12.6 (±92.9) | 15.9 (±31.8) | 13.5 (±139) |
| Total power | 1.93e3 (±4.82e6) | 3.36e3 (±9.99e6) | 49.7 (±2.75e3) | 67 (±4.86e3) |
| (ms²) | | | | |
| VLF power | 965 (±2.11e6) | 880 (±3.52e5) | 32.2 (±1.21e3) | 38.1 (±2.49e3) |
| (ms²) | | | | |
| LF power (ms²) | 426 (±1.29e5) | 658 (±4.25e5) | 8.51 (±89.2) | 15.9 (±233) |
| HF power (ms²) | 368 (±3.84e5) | 1.67e3 (±4.13e6) | 5.6 (±120) | 6.27 (±82.7) |
| VLF norm | 45 (±292) | 34.7 (±324) | 61.6(±123) | 53.6 (±280) |
| (n.u.) | | | | |
| LF norm (n.u.) | 28.2 (±207) | 20.7 (±89.7) | 19.6 (±52.3) | 25 (±224) |
| HF norm (n.u.) | 19.9 (±259) | 40.3 (±249) | 11.8 (±50.6) | 11.4 (±102) |
| LF/HF (n.u.) | 2.4 (±5.34) | 0.606 (±0.121) | 2.26 (±1.67) | 3.01 (±3.04) |
| LF peak (Hz) | 8.04e−2(±4.81e−4) | 0.144(±1.96e−3) | 0.133 (±1.44e−3) | 0.281 (±1.64e−2) |
| HF peak (Hz) | 0.259 (±6.77e−3) | 0.369 (±9.88e−3) | 0.664 (±3.43e−2) | 2.01 (±0.468) |
| beta (n.u.) | −0.783 (±0.613) | −1.04 (±0.513) | −0.661 (±0.325) | −1.31 (±0.42) |
| SampEn (n.u.) | 1.41 (±0.21) | 1.43 (±9.32e−2) | 1.09 (±0.204) | 0.938 (±0.21) |
| SD1 (ms) | 26.4 (±262) | 53.1 (±1.44e3) | 3.1 (±2.14) | 3.8 (±7.05) |
| SD2 (ms) | 78.4 (±1.38e3) | 90.6 (±1.09e3) | 13.9 (±41.2) | 14.3 (±59.7) |
| alpha1 (n.u.) | 1.18 (±7.21e−2) | 0.877 (±3.95e−2) | 1.27 (±3.59e−2) | 1.07 (±8.62e−2) |
| alpha2 (n.u.) | 0.837 (±5.78e−2) | 0.882 (±5.16e−2) | 1.06 (±2.18e−2) | 1.09 (±2.09e−2) |

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A system comprising:
at least one hardware processor; and
a non-transitory computer-readable storage medium having stored thereon program instructions, the program instructions executable by the at least one hardware processor to:
receive, as input, a signal representing temporal beat activity in a mammalian,
receive indication of user selection of an integration level of said beat rate activity,
receive indication of user selection of a species of said mammalian, and
determine heart rate variation (HRV) in said signal, based, at least in part, on said integration level and said species of said mammalian.

2. The system of claim 1, wherein said integration level is selected from the group consisting of: In vivo heart beat rate, sinoatrial node tissue beat rate, and sinoatrial node cell beat rate.

3. The system of claim 1, wherein said signal is at least one of an action potential signal, an electrogram signal, an electrocardiograph (ECG) signal, and a photoplethysmogram (PPG) signal.

4. (canceled)

5. The system of claim 1, wherein said determining is based, at least in part, on one or more HRV parameters associated with said species of said mammalian.

6. (canceled)

7. The system of claim 5, wherein said HRV parameters are selected from the group consisting of: pNNxx threshold, very low frequency (VLF) band, low frequency (LF) band, high frequency (HF) band, and window size for power spectral density (PSD).

8. The system of claim 5, wherein at least some of said HRV parameters associated with said species of said mammalian are retrieved from a database of said HRV parameters associated with a plurality of mammalian species; or wherein at least some of said HRV parameters with respect to a mammalian species are calculated based, at least in part, on an empirical relationship between a mean heart rate value for said species and a power spectral density analysis of said beat rate activity.

9. (canceled)

10. The system of claim 1, wherein said determining comprises first detecting R-peaks in said signal, based, at least in part, on detecting the R-peak location within a specified portion of a duration of each QRS complex in said signal, and wherein said detecting takes into account one or more physical parameters associated with a species of said mammalian.

11. The system of claim 10, wherein said specified portion is between 75% and 85% of an average duration of said QRS complex associated with said species of said mammalian; or wherein said physical parameters are selected from the group consisting of: Mean heart rate, mean QRS duration, mean QT interval duration, minimum R-peak-to-R-peak interval, maximum R-peak-to-R-peak interval, typical QRS peak-to-peak amplitude, and minimum QRS peak-to-peak amplitude.

12. (canceled)

13. The system of claim 10, wherein said detecting further comprises filtering said signal to remove all said beats associated with non-normal sinus node polarizations.

14. (canceled)

15. A method comprising:
operating at least one hardware processor for executing a genetic-type machine learning algorithm configured for:
a non-transitory computer-readable storage medium having stored thereon program instructions, the program instructions executable by the at least one hardware processor to:
receiving, as input, a signal representing temporal beat activity in a mammalian,
receiving indication of user selection of an integration level of said beat rate activity,
receiving indication of user selection of a species of said mammalian, and
determining HRV in said signal, based, at least in part, on said integration level and said species of said mammalian.

16. The method of claim 15, wherein said integration level is selected from the group consisting of: in vivo heart beat rate, sinoatrial node tissue beat rate, and sinoatrial node cell beat rate.

17. The method of claim 15 16, wherein said signal is at least one of an action potential signal, an electrogram signal, an electrocardiograph (ECG) signal, and a photoplethysmogram (PPG) signal.

18. (canceled)

19. The method of claim 15, wherein said determining is based, at least in part, on one or more HRV parameters associated with said species of said mammalian.

20. The method of claim 19, wherein one or more values associated with each of said parameters is adapted based, at least in part, on said species of said mammalian.

21. The method of claim 19, wherein said HRV parameters are selected from the group consisting of: pNNxx threshold, very low frequency (VLF) band, low frequency (LF) band, high frequency (HF) band, and window size for power spectral density (PSD).

22. The method of claim 19, wherein at least some of said HRV parameters associated with said species of said mammalian are retrieved from a database of said HRV parameters associated with a plurality of mammalian species; or wherein at least some of said HRV parameters with respect to a mammalian species are calculated based, at least in part, on an empirical relationship between a mean heart rate value for said species and a power spectral density analysis of said beat rate activity.

23. (canceled)

24. The method of claim 15, wherein said determining comprises first detecting R-peaks in said signal, based, at least in part, on detecting the R-peak location within a specified portion of a duration of each QRS complex in said signal, and wherein said detecting takes into account one or more physical parameters associated with a species of said mammalian.

25. (canceled)

26. The method of claim 24, wherein said physical parameters are selected from the group consisting of: Mean heart rate, mean QRS duration, mean QT interval duration, minimum R-peak-to-R-peak interval, maximum R-peak-to-R-peak interval, typical QRS peak-to-peak amplitude, and minimum QRS peak-to-peak amplitude or wherein said detecting further comprises filtering said signal to remove all said beats associated with non-normal sinus node polarizations.

27. (canceled)

28. (canceled)

29. A computer program product comprising a non-transitory computer-readable storage medium having program instructions embodied therewith, the program instructions executable by at least one hardware processor to execute a genetic-type machine learning algorithm configured for:
receiving, as input, a signal representing temporal beat activity in a mammalian,
receiving indication of user selection of an integration level of said beat rate activity,
receiving indication of user selection of a species of said mammalian, and
determining HRV in said signal, based, at least in part, on said integration level and said species of said mammalian.

30. The computer program product of claim 29, wherein said integration level is selected from the group consisting of: in vivo heart beat rate, sinoatrial node tissue beat rate, and sinoatrial node cell beat rate.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
